(19) United States
(12) Patent Application Publication
(10) Pub. No.: US 2024/0075073 A1
(43) Pub. Date: Mar. 7, 2024

(54) TREATING OPTIC NEURITIS WITH INDUCED PLURIPOTENT STEM CELL-DERIVED OLIGODENDROCYTE PRECURSOR CELLS

(71) Applicant: Mayo Foundation for Medical Education and Research, Rochester, MN (US)

(72) Inventors: Charles L. Howe, Rochester, MN (US); Miranda M. Standiford, Rochester, MN (US); Kanish Mirchia, Rochester, MN (US)

(73) Assignee: Mayo Foundation for Medical Education and Research, Rochester, MN (US)

(21) Appl. No.: 18/217,160

(22) Filed: Jun. 30, 2023

Related U.S. Application Data
(62) Division of application No. 16/091,293, filed on May 19, 2019, now abandoned.
(60) Provisional application No. 62/317,839, filed on Apr. 4, 2016.

(51) Int. Cl. A61K 35/30; A61K 9/00; C12N 5/079; G01N 33/50

(52) U.S. Cl. CPC A61K 35/30; A61K 9/0019; A61K 9/0048; C12N 5/0622; G01N 33/5058; C12N 2502/08

(57) ABSTRACT

This document provides materials and methods for treating a damaged optic nerve in a mammal to restore visual function comprising administering a population of induced pluripotent stem cell-derived oligodendrocyte precursor cells. This document also provides materials and methods for determining a remyelination potential quotient of a population of induced pluripotent stem cell-derived oligodendrocyte precursor cells. This document also provides materials and methods for screening factors that enhance maturation or myelination efficiency of an induced pluripotent stem cell-derived oligodendrocyte precursor cell or cells.

### CROSS-REFERENCE TO RELATED APPLICATIONS

This application is a Divisional of U.S. application Ser. No. 16/091,293, filed on May 19, 2019, which is a National Stage application under 35 U.S.C. § 371 of International Application No. PCT/US2017/025701, having an International Filing Date of Apr. 3, 2017, which claims priority to U.S. Provisional Application Ser. No. 62/317,839, filed on Apr. 4, 2016. The disclosure of the prior application is applications are considered part of the disclosure of this application, and are incorporated in their entirety into this application.

### BACKGROUND

### 1. Technical Field

This document provides materials and methods for treating a damaged optic nerve in a mammal comprising administering a population of induced pluripotent stem cell-derived oligodendrocyte precursor cells, materials and methods for determining a remyelination potential quotient of a population of induced pluripotent stem cell-derived oligodendrocyte precursor cells, and materials and methods for screening for factors that enhance maturation or myelination efficiency of an induced pluripotent stem cell-derived oligodendrocyte precursor cell or cells.

### 2. Background Information

Across the United States, approximately 400,000 people have multiple sclerosis (MS), and according to the National Multiple Sclerosis Society approximately 200 new cases are diagnosed every week. Many of these cases involve young people (18-45 years of age), especially females, struck at the peak of their economic and societal productivity. Optic neuritis is the heralding symptom in 15-20% of patients with MS and is associated with a 30-fold increase in the risk of developing MS. Nearly 80% of MS patients experience optic neuritis during the disease, with unilateral or bilateral attacks that result in permanent reduction or loss of vision in one or both eyes in 40-60% of patients. While some patients recover central vision, one third of affected eyes exhibit persistent visual impairment that includes reduced contrast sensitivity and problems with motion processing and depth perception. Almost half of patients with transient optic neuritis have a recurrent attack within 10 years. Median visual acuity of the affected eye in patients with optic neuritis is 20/60 and optic nerve atrophy can be measured within weeks following onset of the inflammatory attack. Moreover, the prevalence of persistent visual complaints in MS patients has been estimated at around 35%, and even MS patients without a clinical history of optic neuritis exhibit poor visual function, including diffuse visual field defects, reduced low-contrast acuity, impaired depth and motion perception, and decreased color-sensitivity. Pathophysiologically, restoration of vision in these patients requires preservation of the optic nerve axons and repair of the myelin sheath that confers such protection and facilitates high-speed, coordinated impulse conduction from the retina to higher visual processing centers within the brain.

Currently, the therapies that exist to treat patients with demyelinating optic neuritis are purely palliative and almost exclusively immunomodulatory.

### SUMMARY

This document provides materials and methods for treating a damaged optic nerve in a mammal. For example, this document provides materials and methods for identifying a population of induced pluripotent stem cell-derived (iPSC-derived) oligodendrocyte precursor cells (OPCs) as having a remyelination potential quotient (RPQ) greater than about 25% (e.g., more than 1 in 4 cells acquire a phenotype of a mature myelinating oligodendrocyte) and administering the population of induced pluripotent stem cell-derived oligodendrocyte precursor cells, or portion thereof, to the mammal. As described herein, a mammal having a damaged optic nerve can be effectively treated with a population of induced pluripotent stem cell-derived oligodendrocyte precursor cells having a sufficiently high remyelination potential quotient.

In general, one aspect of this document features a method for treating a damaged optic nerve in a mammal. The method comprises, or consist essentially of, (a) identifying said mammal as having a condition of the optic nerve comprising optic nerve demyelination, (b) identifying a population of induced pluripotent stem cell-derived oligodendrocyte precursor cells as having a remyelination potential quotient greater than about 25 percent, and (c) administering said population of induced pluripotent stem cell-derived oligodendrocyte precursor cells to said mammal. The mammal can be a human. The population can be identified as having a remyelination potential quotient greater than about 25 percent by culturing a first portion of the population of induced pluripotent stem cell-derived oligodendrocyte precursor cells in a microfluidic device comprising first and second microfluidic chambers, wherein the first microfluidic chamber comprises a neuron cell body of a cortical neuron, wherein the second microfluidic chamber comprises an axon of the cortical neuron, and wherein the first portion of the population of induced pluripotent stem cell-derived oligodendrocyte precursor cells is co-cultured with the axon in the second microfluidic chamber. The population can be identified as having a remyelination potential quotient greater than about 25 percent by determining the number of cells of the first portion of the population of induced pluripotent stem cell-derived oligodendrocyte precursor cells having a characteristic of a mature, myelinating oligodendrocyte and dividing the number of cells of the first portion by the number of induced pluripotent stem cell-derived oligodendrocyte precursor cells introduced into the second microfluidic chamber. The characteristic of a mature, myelinating oligodendrocyte can be selected from the group consisting of: a morphological characteristic, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, expression of a GST-pi mRNA, and combinations thereof. The remyelination potential quotient can be determined to be sufficient for administration of the population of induced pluripotent stem cell-derived oligodendrocyte precursor cells to the mammal if the remyelination potential quotient is about 30 percent or higher.

The microfluidic device can further comprise a third microfluidic chamber, wherein the second microfluidic chamber comprises a segment of said axon, wherein the third microfluidic chamber comprises a distal end of the axon, and wherein a second portion of the population of induced pluripotent stem cell-derived oligodendrocyte precursor cells is co-cultured with the distal end of the axon in the third microfluidic chamber. The population of induced pluripotent stem cell-derived oligodendrocyte precursor cells can be identified as having a remyelination potential quotient greater than about 25 percent by determining the number of cells of the first portion of the population of induced pluripotent stem cell-derived oligodendrocyte precursor cells having a characteristic of a mature, myelinating oligodendrocyte and dividing the number of cells of the first portion by the number of induced pluripotent stem cell-derived oligodendrocyte precursor cells introduced into the second microfluidic chamber. Additionally or alternatively, the population of induced pluripotent stem cell-derived oligodendrocyte precursor cells can be determined to have a remyelination potential quotient greater than about 25 percent by determining the number of cells of the second portion of the population of induced pluripotent stem cell-derived oligodendrocyte precursor cells having a characteristic of a mature, myelinating oligodendrocyte and dividing the number of cells of the second portion by the number of induced pluripotent stem cell-derived oligodendrocyte precursor cells introduced into the third microfluidic chamber. The characteristic of a mature, myelinating oligodendrocyte can be selected from the group consisting of: a morphological characteristic, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, expression of a GST-pi mRNA, and combinations thereof. The remyelination potential quotient can be determined to be sufficient for administration of the population of induced pluripotent stem cell-derived oligodendrocyte precursor cells to the mammal if the remyelination potential quotient is about 30 percent or higher.

Administering can comprise intravitreal injection of the population of induced pluripotent stem cell-derived oligodendrocyte precursor cells. The mammal can have a condition comprising multiple sclerosis, demyelinating optic neuritis, or both. Administering can drive remyelination of the optic nerve, restore axonal conduction, or both.

Another aspect of this document features a method for determining a remyelination potential quotient of a population of induced pluripotent stem cell-derived oligodendrocyte precursor cells. The method comprises, or consist essentially of, culturing a first portion of the population of induced pluripotent stem cell-derived oligodendrocyte precursor cells in a microfluidic device comprising first and second microfluidic chambers, wherein the first microfluidic chamber comprises a neuron cell body of a cortical neuron, wherein the second microfluidic chamber comprises an axon of the cortical neuron, and wherein the first portion of the population of induced pluripotent stem cell-derived oligodendrocyte precursor cells is co-cultured with the axon in the second microfluidic chamber. The remyelination potential quotient can be determined by determining the number of cells of the first portion of the population of induced pluripotent stem cell-derived oligodendrocyte precursor cells having a characteristic of a mature, myelinating oligodendrocyte and dividing the number of cells of the first portion by the number of induced pluripotent stem cell-derived oligodendrocyte precursor cells introduced into the second microfluidic chamber. The characteristic of a mature, myelinating oligodendrocyte can be selected from the group consisting of: a morphological characteristic, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, expression of a GST-pi mRNA, and combinations thereof. The remyelination potential quotient can be determined to be sufficient for administration of the population of induced pluripotent stem cell-derived oligodendrocyte precursor cells to the mammal if the remyelination potential quotient is about 30 percent or higher.

The microfluidic device can further comprise a third microfluidic chamber, wherein the second microfluidic chamber comprises a segment of the axon, wherein the third microfluidic chamber comprises a distal end of the axon, and wherein a second portion of the population of induced pluripotent stem cell-derived oligodendrocyte precursor cells is co-cultured with the distal end of the axon in the third microfluidic chamber. The remyelination potential quotient can be determined by determining the number of cells of the first portion of the population of induced pluripotent stem cell-derived oligodendrocyte precursor cells having a characteristic of a mature, myelinating oligodendrocyte and dividing the number of cells of the first portion by the number of induced pluripotent stem cell-derived oligodendrocyte precursor cells introduced into the second microfluidic chamber. Additionally or alternatively, the remyelination potential quotient can be determined by determining the number of cells of the second portion of the population of induced pluripotent stem cell-derived oligodendrocyte precursor cells having a characteristic of a mature, myelinating oligodendrocyte and dividing the number of cells of the second portion by the number of induced pluripotent stem cell-derived oligodendrocyte precursor cells introduced into the third microfluidic chamber. The characteristic of a mature, myelinating oligodendrocyte can be selected from the group consisting of: a morphological characteristic, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, expression of a GST-pi mRNA, and combinations thereof. The remyelination potential quotient can be determined to be sufficient for administration of the population of induced pluripotent stem cell-derived oligodendrocyte precursor cells to the mammal if the remyelination potential quotient is about 25 percent or above.

Another aspect of this document features a method for screening for factors that enhance maturation or myelination efficiency of an induced pluripotent stem cell-derived oligodendrocyte precursor cell. The method comprises, or consist essentially of, culturing the induced pluripotent stem cell-derived oligodendrocyte precursor cell in a microfluidic device comprising first and second microfluidic chambers, wherein the first microfluidic chamber comprises a neuron cell body of a cortical neuron, wherein the second microfluidic chamber comprises an axon of the cortical neuron, wherein the induced pluripotent stem cell-derived oligodendrocyte precursor cell is co-cultured with the axon in the second microfluidic chamber, providing a first test factor to the second microfluidic chamber, and determining the maturation or myelination efficiency of the induced pluripotent stem cell-derived oligodendrocyte precursor cell in the second microfluidic chamber. The microfluidic device can further comprise a third microfluidic chamber, wherein the second microfluidic chamber comprises a segment of the axon, wherein the third microfluidic chamber comprises a distal end of the axon, and wherein a second induced pluripotent stem cell-derived oligodendrocyte precursor cell is co-cultured with the distal end of the axon in the third microfluidic chamber, providing a second test factor to the third microfluidic chamber, and determining the maturation or myelination efficiency of the induced pluripotent stem cell-derived oligodendrocyte precursor cell in the third microfluidic chamber. The maturation or myelination efficiency can be determined by determining a characteristic of a mature, myelinating oligodendrocyte selected from the group consisting of: a morphological characteristic, a functional characteristic, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, expression of a GST-pi mRNA, and combinations thereof. The maturation or myelination efficiency of an induced pluripotent stem cell-derived oligodendrocyte precursor cell cultured in the presence of the factor can be increased compared to the maturation or myelination efficiency of an induced pluripotent stem cell-derived oligodendrocyte precursor cell cultured in the absence of the factor.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-1C. Microfluidic chamber design. FIG. 1A) Schematic of a 3-chamber microfluidic device that permits physical separation of neuron cell bodies and axons (irregular diamonds and lines extending from them) while allowing the addition of oligodendrocyte precursor cells OPCs (circles). FIG. 1B) Cortical neurons in a 3-chamber system were transduced with AAV9.hSyn.TurboRFP.WPRE.rBG to drive red fluorescent protein (RFP) expression only in neurons, robustly labeling axons till the distal chamber. An exemplary axon is indicated. OPCs (examples indicated by arrows) were co-cultured with the axons and are shown interacting with neuronal axons in vitro. FIG. 1C) Schematic top-down view and side-view of a two-chamber design. Primary murine cortical neurons were plated in the cell body chamber (CB) and long axons into the distal axon chamber (AX). Micrograph shows neurons and axons growing in the chamber. The cell body chamber was stained with DAPI to show the cell bodies of the axons. (In the original color micrograph: red=neurofilament; blue=DAPI). Scale bar in FIG. 1B=200 μm; scale bar in FIG. 1C=10 μm.

FIGS. 2A-2D. In vitro evidence of OPC maturation. FIG. 2A) Stage-specific markers of oligodendrocyte development. Relevant markers of mature oligodendrocytes are MOG CC1, MPB, and PLP, as highlighted in the shaded box. FIG. 2B) Live imaging of maturing oligodendrocytes (GFP+, green) making numerous contacts with axons. FIG. 2C) OPCs (green) cultured with axons extend processes and wrap fibers. FIG. 2D) Immunostaining confirms production of mature myelin proteins. OPCs, PLP staining, and DAPI staining is indicated by arrows. Scale bars in FIG. 2B, FIG. 2D are 50 μm, scale bar in FIG. 2C is 20 μm.

FIGS. 3A-3F. In vitro evidence of OPC maturation. In vitro evidence of OPC maturation. FIG. 3A) Stage-specific markers of oligodendrocyte development. Relevant markers of mature oligodendrocytes are MOG CC1, MPB, and PLP, as highlighted in the shaded box. FIG. 3B) Longitudinal temporal imaging of OPCs (green, GFP+, exemplary OPCs are indicated by arrowheads) co-cultured with neuronal axons, exhibiting morphological maturation (from left to right). By 11 days in culture, OPCs exhibited the phenotype of a mature oligodendrocyte, making multiple contacts with multiple axons. FIG. 3C) Schematic of 3-chamber microfluidic design to show the maturation pattern exhibited by OPCs in culture in FIG. 3B. FIGS. 3D-3F) Transmission electron micrographs showing FIG. 3D) neuronal cell bodies extending axons in the proximal chamber of the microfluidic device; FIG. 3E) axon bundles passing through microgrooves connecting the proximal and middle chambers; and FIG. 3F) oligodendrocyte extending processes making contact with neuronal axons. Scale bar in FIG. 3D=50 μm, scale bar in FIG. 3E=2 μm, and scale bar in FIG. 3F=5 μm.

FIGS. 4A-4N. Cuprizone-induced demyelination model and live animal assessments. FIGS. 4A-4D) PPD (para-Phenylenediamine) staining of semi-thin (0.6 μm) sections from araldite-embedded optic nerve revealed the pattern of normal myelination in the optic nerve of animals fed control chow (FIGS. 4A-4B) and the profound demyelination observed after 6 weeks of cuprizone treatment (FIGS. 4C-4D). FIGS. 4E-4H) Optical coherence tomography (OCT) provided quantitative assessment of neuroretinal integrity. FIG. 4F) The retinal layers measured along the green line in (FIG. 4E) in a healthy mouse. The clear retinal nerve fiber layer (RNFL) is outlined in FIG. 4H. FIG. 4G) The retinal layers measured along the yellow line in (FIG. 4E) in a healthy mouse. The thick ganglion cell layer (GCL) is outlined in FIG. 4H. FIGS. 4I-4K) Head mount surgery for placement of VEP electrodes and recording locations. FIG. 4L) Visual evoked potentials (VEPs) measured in the visual cortex induced by a 0.3 Hz light pulse. The top trace represents unstimulated potentials, the bottom trace shows the potentials evoked by repetitive stimulation. FIG. 4M) VEP averaging shows measurements of evoked potential amplitude, latency, and pulse width. FIG. 4N) VEP recording from visual cortex shows increased latency to N1 at 0.3 Hz stimulation frequency and increased failure to transmit impulses in the cuprizone-fed animal (bottom trace) compared to animals on regular diet (top trace). Scale bar in FIG. 4C=100 μm and refers to FIG. 4A; scale bar in FIG. 4D=10 μm and refers to FIG. 4B.

FIGS. 5A-5D. Cuprizone-induced demyelination and remyelination after OPC transplant in murine optic nerves. FIGS. 5A-5B) PPD (para-Phenylenediamine staining of semi-thin (0.6 μm) sections from araldite-embedded optic nerves revealed the pattern of normal myelination in the optic nerve of animals fed control chow (FIG. 5B) and the profound demyelination observed after 9 weeks of cuprizone treatment (FIG. 5A). FIGS. 5C-5D) Electron micrographs of thin optic nerve cross sections revealed thinning and disorganization of myelin layers in cuprizone treated animals (FIG. 5C) and restoration of compacted myelin two weeks after OPC transplant injection (FIG. 5D).

FIGS. 6A-6C. Intravitreal transplantation of iPSC-derived OPCs. FIG. 6A) Intravitreal injection scheme. iPSC-derived OPCs were delivered into the vitreous cavity, near the optic nerve head at a concentration of 10⁸ cells/mL in a 1.0 μL volume. FIG. 6B) OPCs (*) producing long green (GFP⁺) myelin fibers (arrowheads) extending ˜0.85 mm from the proximal unmyelinated region of the optic nerve, in a cuprizone-demyelinated animal at 7 days post-transplant (dpt). FIG. 6C) Maximum intensity projection of whole mounted optic nerve from healthy control animal showing migration of GFP+ OPCs (*) up to 1.87 mm away from the eye at 7 dpt.

FIGS. 7A-7J. Functional assessment of remyelination in vivo. FIG. 7A) Surgical apparatus and subject following electrode placement. FIGS. 7B-7C) Schematic representation of electrode placement on skull and wiring setup. FIG. 7D) Visual evoked potential (VEP) averaging was used to establish measurements of evoked potential amplitude, latency, and peak pulse-width. FIGS. 7E-7G) Representative waveforms of VEP recordings in a healthy control animal (FIG. 7E, top), cuprizone-fed animal (FIG. 7F, middle), and cuprizone-fed animal transplanted with OPCs (FIG. 7Q bottom). FIG. 7H) Latency was measured as the time from the visual stimulus to the first negative peak in the VEP impulse. An increased latency indicated decreased conduction velocity as a result of loss of myelin and Nodes of Ranvier. The average latency increased from 276 ms to 371 ms after cuprizone treatment for nine weeks, and normalized to 265 ms after OPC transplant treatment. FIG. 7I) Peak width was measured as the duration of the first positive impulse at half-maximum amplitude. An increase in peak width denoted unsynchronized impulses travelling to the visual cortex at different speeds along axons with differing loss of myelin and conduction velocities. The shortest half for peak width values increased after demyelination with cuprizone, and returned towards baseline after OPC transplant. FIG. 7J) Number of missed responses represents the number of times a fixed stimulus did not evoke a response. Number of missed responses was greatly increased in cuprizone fed animals and re-normalized after OPC transplant. All analysis was completed using MATLAB (MathWorks, MA, USA). n=5 for all groups.

### DETAILED DESCRIPTION

This document provides materials and methods for treating a damaged optic nerve in a mammal. For example, this document provides materials and methods for identifying a population of induced pluripotent stem cell-derived (iPSC-derived) oligodendrocyte precursor cells (OPCs) as having a remyelination potential quotient (RPQ) greater than about 25 percent.

As described herein, a mammal having a damaged optic nerve can be effectively treated with a population of induced pluripotent stem cell-derived oligodendrocyte precursor cells having a sufficiently high RPQ (e.g., an RPQ greater than about 25, 35, 45, 50, 55, 65, or 75 percent). Any appropriate mammal having a damaged optic nerve can be treated as described herein. For example, humans and non-human primates such as monkeys can be identified as having a damaged optic nerve and treated with a population of induced pluripotent stem cell-derived oligodendrocyte precursor cells to drive remyelination of the optic nerve, restore axonal conduction, or both. In some cases, dogs, cats, horses, cows, pigs, sheep, mice, and rats can be identified and treated with a population of induced pluripotent stem cell-derived oligodendrocyte precursor cells as described herein.

Any appropriate type of damage to the optic nerve can be treated in accordance with materials and methods provided herein. For example, damage to the optic nerve to be treated in accordance with materials and methods provided herein in the mammal can be caused by multiple sclerosis (MS). In some cases, the mammal can have a condition comprising multiple sclerosis, demyelinating optic neuritis, or both. In some cases, damage to the optic nerve to be treated in accordance with materials and methods provided herein can be caused by demyelination of the optic nerve induced by infiltrating inflammatory effector cells resulting in transient disruption of axonal conduction followed by chronic slowing, mistiming, and stochastic failure of the visual impulses that are transmitted from the retina to higher-order visual processing centers in order to confer vision. Such demyelinated axons become susceptible to injury and transection mediated by cellular immune effectors, toxic inflammatory mediators, and intra-axonal metabolic dysregulation, resulting in permanent and irrecoverable loss of information transmission through the visual pathway.

Any appropriate method can be used to identify a mammal having a damaged optic nerve. For example, imaging techniques, techniques to test and analyze vision, and visual evoked potentials (VEPs) can be used to identify mammals (e.g., humans) having a damaged optic nerve.

In some cases, a remyelination potential quotient of a population of induced pluripotent stem cell-derived oligodendrocyte precursor cells can be determined prior to administering the population to a mammal. In some cases, a remyelination potential quotient of a population of induced pluripotent stem cell-derived oligodendrocyte precursor cells can be determined by culturing the population of induced pluripotent stem cell-derived oligodendrocyte precursor cells, or portion thereof, in a microfluidic device comprising a plurality of microfluidic chambers, e.g. a first and second microfluidic chamber. In some cases, a population of induced pluripotent stem cell-derived oligodendrocyte precursor cells, or portion thereof, can be co-cultured in a microfluidic device with one or more neurons, e.g., one or more cortical neurons. In some cases, a single cortical neuron is cultured in a plurality of chambers of the microfluidic device. For example, a cortical neuron can be cultured in a microfluidic device such that the body of the cortical axon is cultured in one microfluidic chamber and the axon of the cortical neuron is cultured in one or more other microfluidic chambers that can be fluidically separated. In some cases, a cortical neuron can be cultured in a microfluidic device such that the body of the cortical axon is cultured in one microfluidic chamber, an axon is cultured in a second microfluidic chamber, and the distal end of the axon is cultured in third microfluidic chamber. In some cases, a population of induced pluripotent stem cell-derived oligodendrocyte precursor cells, or portion thereof, can be co-cultured with an axon in the same microfluidic chamber. In some cases, a population of induced pluripotent stem cell-derived oligodendrocyte precursor cells, or portion thereof, can be co-cultured en passant of an axon in the same microfluidic chamber. In some cases, a population of induced pluripotent stem cell-derived oligodendrocyte precursor cells, or portion thereof, can be co-cultured with the distal end of an axon in the same microfluidic chamber. By exploiting the microfluidic separation of the cell body chamber from the axon chamber or chambers, it is possible to introduce different media formulations into the separate chambers.

In some cases, a remyelination potential quotient of the population of induced pluripotent stem cell-derived oligodendrocyte precursor cells can be determined by determining the number of cells of a first portion of the population of induced pluripotent stem cell-derived oligodendrocyte precursor cells having a characteristic of a mature, myelinating oligodendrocyte and dividing the number of mature cells of the first portion by the total number of induced pluripotent stem cell-derived oligodendrocyte precursor cells introduced into the same microfluidic chamber. For example, if 80% of the cells of the first portion of the population of induced pluripotent stem cell-derived oligodendrocyte precursor cells have a characteristic of a mature, myelinating oligodendrocyte, the RPQ of the population is 80%. A remyelination potential quotient of a population of induced pluripotent stem cell-derived oligodendrocyte precursor cells can be similarly determined in other embodiments described herein.

In some cases, a microfluidic device used to assess cells capable of effectively treating a damaged optic nerve in a mammal can have three microfluidic chambers. In some cases, a neuron, e.g., a cortical neuron, can be cultured in such a three-chambered microfluidic device. For example, the body of a neuron can be cultured in a first chamber, an axon of the neuron can be cultured in a second chamber, and the distal end of the axon of the neuron can be cultured in a third chamber. In some cases, a population of induced pluripotent stem cell-derived oligodendrocyte precursor cells, or portion thereof, can be co-cultured with: 1) a segment of an axon of the neuron being cultured in the second chamber, 2) the distal end of the axon of the neuron being cultured in the third chamber, or 3) both. In some cases, the remyelination potential quotient of a population of induced pluripotent stem cell-derived oligodendrocyte precursor cells can be determined by determining the number of cells of the portion of the population co-cultured en passant of the axon being cultured having a characteristic of a mature, myelinating oligodendrocyte and dividing that number by the total number of induced pluripotent stem cell-derived oligodendrocyte precursor cells introduced into the second microfluidic chamber. In some cases, the remyelination potential quotient of a population of induced pluripotent stem cell-derived oligodendrocyte precursor cells can be determined by determining the number of cells of the portion of the population co-cultured with a distal end of an axon of the neuron being cultured having a characteristic of a mature, myelinating oligodendrocyte and dividing that number by the total number of induced pluripotent stem cell-derived oligodendrocyte precursor cells introduced into the third microfluidic chamber.

Any suitable characteristic or characteristics of mature, myelinating oligodendrocytes can be used to determine the remyelination potential quotient of a population of induced pluripotent stem cell-derived oligodendrocyte precursor cells. Examples of such characteristics include, without limitation, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, and expression of a GST-pi mRNA. In some cases, a characteristic of a mature, myelinating oligodendrocyte can be a morphological characteristic. For example, a morphological characteristic of a mature, myelinating oligodendrocyte can be the making of one or more contacts with one or more axons of a cortical neuron, and branched morphology with axon ensheathment. In some cases, a characteristic of a mature, myelinating oligodendrocyte can be a functional characteristic. For example, a functional characteristic of a mature, myelinating oligodendrocyte can be myelinating or remyelinating activity on an axon of a cortical neuron. In some cases, the remyelination potential quotient of a population of induced pluripotent stem cell-derived oligodendrocyte precursor cells can be determined by determining the number of cells of a population, or portion thereof, having one or more characteristics selected from a plurality of characteristics of a mature, myelinating oligodendrocyte, e.g. an expression characteristic or characteristics, a morphological characteristic or characteristics, and/or a functional characteristic or characteristics of a mature, myelinating oligodendrocyte.

In some cases, a remyelination potential quotient of a population of induced pluripotent stem cell-derived oligodendrocyte precursor cells can be determined to be sufficient for administration of the population to the mammal if the remyelination potential quotient is above a certain threshold. For example, suitable remyelination potential quotient threshold for a population of induced pluripotent stem cell-derived oligodendrocyte precursor cells can be greater than 25%, 30%, 35%, 40%, 45%, 50%, 55%, 60%, 65%70%, 75%, 80%, 85%, 90%, or 95%. In general, the higher the RPQ of a population of induced pluripotent stem cell-derived oligodendrocyte precursor cells, the more suitable that population is for administration to a mammal.

In some cases, a remyelination potential quotient of a population of induced pluripotent stem cell-derived oligodendrocyte precursor cells for use in treating a damaged optic nerve of a mammal (e.g., a human) can be determined by determining the remyelination potential quotient of two or more subpopulations of the population of induced pluripotent stem cell-derived oligodendrocyte precursor cells according to one or more methods described herein. For example, a remyelination potential quotient of a population of induced pluripotent stem cell-derived oligodendrocyte precursor cells can be determined by determining the RPQ of 2, 3, 4, 5, 6, 7, 8, 9, 10, or more subpopulations. In some cases, a RPQ of a population of induced pluripotent stem cell-derived oligodendrocyte precursor cells can be determined by determining the RPQ of two or more subpopulations using two or more microfluidic devices as described herein, e.g., two or more microfluidic devices having three microfluidic chambers, wherein a neuron, e.g., a cortical neuron, is co-cultured in the two or more microfluidic devices with the two or more subpopulations of the population of induced pluripotent stem cell-derived oligodendrocyte precursor cells. In some cases, a remyelination potential quotient of a population of induced pluripotent stem cell-derived oligodendrocyte precursor cells can be determined by co-culturing two or more subpopulations of the population in two or more microfluidic devices, wherein each of the subpopulations is co-cultured en passant of an axon, e.g., an axon of a cortical neuron. In some cases, a remyelination potential quotient of a population of induced pluripotent stem cell-derived oligodendrocyte precursor cells can be determined by co-culturing two or more subpopulations of the population in two or more microfluidic devices, wherein each of the subpopulations is co-cultured with a distal end of a neuron, e.g., a cortical neuron. In some cases, a remyelination potential quotient of a population of induced pluripotent stem cell-derived oligodendrocyte precursor cells can be determined by averaging the determined remyelination potential quotients of two or more subpopulations of the population. In some cases, a coefficient of variance for log-normalized remyelination potential quotient values of each subpopulation can be calculated as a measure of reproducibility.

In some cases, RPQ of a population of induced pluripotent stem cell-derived oligodendrocyte precursor cells can be clonally derived from a single pluripotent stem cell-derived oligodendrocyte precursor cell. In some cases, a population of induced pluripotent stem cell-derived oligodendrocyte precursor cells can be derived from heterogeneous starting population of a pluripotent stem cell-derived oligodendrocyte precursor cells.

In some cases, once a population of induced pluripotent stem cell-derived oligodendrocyte precursor cells is determined to have a suitable remyelination potential quotient, aliquots of that population can be frozen and stored for future use.

Any suitable route of administration can be used in accordance with methods of treating a damaged optic nerve in a mammal, as provided herein. In some cases, a population of induced pluripotent stem cell-derived oligodendrocyte precursor cells can be administered to the mammal via an intravitreal injection, e.g. injection into the vitreous proximal to the optic nerve head. A population of induced pluripotent stem cell-derived oligodendrocyte precursor cells to be injected into a mammal can be formulated with any number of acceptable carriers, fillers, and/or vehicles.

Effective numbers of induced pluripotent stem cell-derived oligodendrocyte precursor cells in an administered population can vary depending on the severity of the damage to the optic nerve, the route of administration, the age and general health condition of the subject, excipient usage, the possibility of co-usage with other therapeutic treatments such as use of other agents, and the judgment of the treating physician. In some cases, from about 1×10⁷ to about 5×10⁷ cells/eye can be administered to a mammal (e.g., a human). In some cases, from about 10⁷ to about 10⁸, from about 10⁶ to about 10⁷, from about 10⁵ to about 10⁶, from about 10⁴ to about 10⁵, from about 10³ to about 10⁴ cells/eye can be administered to a mammal (e.g., a human).

If a particular mammal fails to respond to a particular number of induced pluripotent stem cell-derived oligodendrocyte precursor cells, the number of induced pluripotent stem cell-derived oligodendrocyte precursor cells can be increased. In some cases, the number of induced pluripotent stem cell-derived oligodendrocyte precursor cells can be increased by 2-fold, 3-fold, 4-fold, 5-fold, 10-fold, or more. After receiving such an increased number of induced pluripotent stem cell-derived oligodendrocyte precursor cells, the mammal can be monitored for both responsiveness to the treatment and toxicity symptoms, and adjustments made accordingly. The effective number of induced pluripotent stem cell-derived oligodendrocyte precursor cells can remain constant or can be adjusted as a sliding scale or variable numbers depending on the mammal's response to treatment. Various factors can influence the actual effective number of induced pluripotent stem cell-derived oligodendrocyte precursor cells used. For example, the frequency of administration, duration of treatment, use of multiple treatment agents, route of administration, and severity of the condition (e.g., damage to an optic nerve) may require an increase or decrease in the actual effective number of induced pluripotent stem cell-derived oligodendrocyte precursor cells administered.

In some cases, for humans, a population of autologous induced pluripotent stem cell-derived oligodendrocyte precursor cells can be administered. For non-human mammals, a population of autologous or homologous induced pluripotent stem cell-derived oligodendrocyte precursor cells can be administered.

In some cases, a population of induced pluripotent stem cell-derived oligodendrocyte precursor cells can be administered once to a mammal. In some cases, a population of induced pluripotent stem cell-derived oligodendrocyte precursor cells can be administered more than once to a mammal.

In some cases, a population of induced pluripotent stem cell-derived oligodendrocyte precursor cells can be administered to a mammal (e.g., a human) in combination with one or more additional therapeutic agents. In some cases, a population of induced pluripotent stem cell-derived oligodendrocyte precursor cells can be administered to a mammal in combination with valproic acid. In some cases, a combination therapy of a population of induced pluripotent stem cell-derived oligodendrocyte precursor cells and valproic acid can enhance OPC recruitment, survival, and/or cumulative myelination compared to either therapy alone. In some cases, such one or more additional therapeutic agents can be blood-brain barrier permeable drugs. Examples of suitable blood-brain barrier permeable drugs include, without limitation, miconazole, clobetasol, and benztropine. In some cases, a population of induced pluripotent stem cell-derived oligodendrocyte precursor cells can be administered to a mammal simultaneously with one or more additional therapeutic agents. In some cases, a population of induced pluripotent stem cell-derived oligodendrocyte precursor cells can be administered to a mammal prior to administration of one or more additional therapeutic agents. In some cases, a population of induced pluripotent stem cell-derived oligodendrocyte precursor cells can be administered to a mammal after administration of one or more additional therapeutic agents.

In some cases, a course of treatment, the damage to or function of an optic nerve present within a mammal, and/or the severity of one or more symptoms related to the condition being treated (e.g., damage to an optic nerve) can be monitored. Any appropriate method can be used to determine whether or not damage to an optic nerve of a mammal is reduced and/or whether the function of the optic nerve is improved. For example, imaging techniques, techniques to test and analyze vision, and visual evoked potentials (VEPs) can be used to determine whether or not damage to an optic nerve of a mammal (e.g., a human) is reduced and/or whether the function of the optic nerve of a mammal (e.g., a human) is improved. In some cases, one or more of evoked potential amplitude, latency, peak pulse-width, and/or number of missed responses can be measured and used to determine whether or not damage to an optic nerve of a mammal is reduced and/or whether the function of the optic nerve is improved. In some cases, treating a mammal (e.g., a human) in accordance with a method provided herein can drive remyelination of the optic nerve, restore axonal health, or both.

This document also provides materials and methods for determining a remyelination potential quotient of a population of induced pluripotent stem cell-derived oligodendrocyte precursor cells. For example, this document provides materials and methods for culturing the population of induced pluripotent stem cell-derived oligodendrocyte precursor cells, or a portion thereof, in a microfluidic device comprising a plurality of microfluidic chambers. As described herein, the population of induced pluripotent stem cell-derived oligodendrocyte precursor cells, or portion thereof, can be co-cultured in one or more of the plurality of microfluidic chambers with an axon of a neuron, e.g., a cortical neuron, and the remyelination potential quotient of the population or portion can be determined.

Any suitable device (e.g., a microfluidic device) or method described herein of determining a remyelination potential quotient of a population of induced pluripotent stem cell-derived oligodendrocyte precursor cells for treating a damaged optic nerve in a mammal can be used to determine a remyelination potential quotient of a population of induced pluripotent stem cell-derived oligodendrocyte precursor cells generally. For example, a population of induced pluripotent stem cell-derived oligodendrocyte precursor cells, or portion thereof, can be co-cultured with a neuron, e.g., a cortical neuron, in a microfluidic device comprising three chambers. For example, the body of a neuron can be cultured in a first chamber, an axon of the neuron can be cultured in a second chamber, and the distal end of the axon of the neuron can be cultured in a third chamber. In some cases, a population of induced pluripotent stem cell-derived oligodendrocyte precursor cells, or portion thereof, can be co-cultured with: 1) an axon of the neuron being cultured in the second chamber, 2) the distal end of the axon of the neuron being cultured in the third chamber, or 3) both. In some cases, the remyelination potential quotient of a population of induced pluripotent stem cell-derived oligodendrocyte precursor cells can be determined by determining the number of cells of the portion of the population co-cultured en passant of an axon being cultured having a characteristic of a mature, myelinating oligodendrocyte and dividing that number by the total number of induced pluripotent stem cell-derived oligodendrocyte precursor cells introduced into the second microfluidic chamber. In some cases, the remyelination potential quotient of a population of induced pluripotent stem cell-derived oligodendrocyte precursor cells can be determined by determining the number of cells of the portion of the population co-cultured with a distal end of an axon of the neuron being cultured having a characteristic of a mature, myelinating oligodendrocyte and dividing that number by the total number of induced pluripotent stem cell-derived oligodendrocyte precursor cells introduced into the third microfluidic chamber.

In some cases, a remyelination potential quotient of a population of induced pluripotent stem cell-derived oligodendrocyte precursor cells can be determined by determining the remyelination potential quotient of two or more subpopulations of the population of induced pluripotent stem cell-derived oligodendrocyte precursor cells according to more or more methods described herein. For example, a remyelination potential quotient of a population of induced pluripotent stem cell-derived oligodendrocyte precursor cells can be determined by determining the remyelination potential quotient of 2, 3, 4, 5, 6, 7, 8, 9, 10, or more subpopulations. In some cases, a remyelination potential quotient of a population of induced pluripotent stem cell-derived oligodendrocyte precursor cells can be determined by determining the remyelination potential quotient of two or more subpopulations using two or more microfluidic devices as described herein, e.g., two or more microfluidic devices having three microfluidic chambers, wherein a neuron, e.g., a cortical neuron, is co-cultured in the microfluidic device with the two or more subpopulations of the population of induced pluripotent stem cell-derived oligodendrocyte precursor cells. In some cases, a remyelination potential quotient of a population of induced pluripotent stem cell-derived oligodendrocyte precursor cells can be determined by co-culturing two or more subpopulations of the population in two or more microfluidic devices, wherein each of the subpopulations is co-cultured en passant of an axon. In some cases, a remyelination potential quotient of a population of induced pluripotent stem cell-derived oligodendrocyte precursor cells can be determined by co-culturing two or more subpopulations of the population in two or more microfluidic devices, wherein each of the subpopulations is co-cultured with a distal end of a neuron, e.g., a cortical neuron. In some cases, a remyelination potential quotient of a population of induced pluripotent stem cell-derived oligodendrocyte precursor cells can be determined by averaging the determined remyelination potential quotient of two or more subpopulations of the population. In some cases, a coefficient of variance for log-normalized remyelination potential quotient values of each subpopulation can be calculated as a measure of reproducibility.

In some cases, a remyelination potential quotient of a population of induced pluripotent stem cell-derived oligodendrocyte precursor cells can be determined to be sufficient for administration of the population of induced pluripotent stem cell-derived oligodendrocyte precursor cells to the mammal if the remyelination potential quotient is above a certain threshold. For example, suitable remyelination potential quotient threshold for a population of induced pluripotent stem cell-derived oligodendrocyte precursor cells can be greater than 25%, 30%, 35%, 40%, 45%, 50%, 55%, 60%, 65%70%, 75%, 80%, 85%, 90%, or 95%. In general, the higher the remyelination potential quotient of a population of induced pluripotent stem cell-derived oligodendrocyte precursor cells, the more suitable that population is for administration to a mammal.

This document also provides materials and methods for screening for factors that enhance maturation or myelination efficiency of an induced pluripotent stem cell-derived oligodendrocyte precursor cell or a population of induced pluripotent stem cell-derived oligodendrocyte precursor cells. For example, this document provides materials and methods for culturing the population of induced pluripotent stem cell-derived oligodendrocyte precursor cells, or a portion thereof, in a microfluidic device comprising a plurality of microfluidic chambers. As described herein, an induced pluripotent stem cell-derived oligodendrocyte precursor cell or cells can be co-cultured in one of the plurality of microfluidic chambers with an axon (e.g., en passant of the axon, distal end of the axon, or both of a neuron, e.g., a cortical neuron, a test factor can be provided to that chamber, and the effect of the test compound on maturation or myelination efficiency of the induced pluripotent stem cell-derived oligodendrocyte precursor cell or cells can be determined.

Any microfluidic device or method of using such a device described herein can be used to screen for factors that enhance maturation or myelination efficiency of an induced pluripotent stem cell-derived oligodendrocyte precursor cell or a population of induced pluripotent stem cell-derived oligodendrocyte precursor cells.

In some cases, a single factor can be screened for its effect and/or its enhancement of maturation or myelination efficiency of an induced pluripotent stem cell-derived oligodendrocyte precursor cell or a population of induced pluripotent stem cell-derived oligodendrocyte precursor cells. In some cases, a plurality of factors can be screened for their effect and/or their enhancement of maturation or myelination efficiency of an induced pluripotent stem cell-derived oligodendrocyte precursor cell or a population of induced pluripotent stem cell-derived oligodendrocyte precursor cells.

Maturation or myelination efficiency can be determined by assaying for any of a variety of characteristics of a mature, myelinating oligodendrocyte. Examples of such characteristics include, without limitation, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, and expression of a GST-pi mRNA. In some cases, a characteristic of a mature, myelinating oligodendrocyte can be a morphological characteristic. For example, a morphological characteristic of a mature, myelinating oligodendrocyte can be the making of one or more contacts with one or more axons of a cortical neuron, and branched morphology with axon ensheathment. In some cases, a characteristic of a mature, myelinating oligodendrocyte can be a functional characteristic. For example, a functional characteristic of a mature, myelinating oligodendrocyte can be myelinating or remyelinating activity on an axon of a cortical neuron. In some cases, a factor (or factors) can be screened for its effect and/or its enhancement of maturation or myelination efficiency of an induced pluripotent stem cell-derived oligodendrocyte precursor cell or a population of induced pluripotent stem cell-derived oligodendrocyte precursor cells by determining whether cells or cells have one or more characteristics selected from a plurality of characteristics of a mature, myelinating oligodendrocyte, e.g. an expression characteristic or characteristics, a morphological characteristic or characteristics, and/or a functional characteristic or characteristics of a mature, myelinating oligodendrocyte.

In some cases, maturation or myelination efficiency can be determined by culturing a population of induced pluripotent stem cell-derived oligodendrocyte precursor cells with one or more factors, and administering the population, or portion thereof, to an animal (e.g., a mouse, a rat, a primate, a non-human mammal, a dog, a cat, a horse, a cow, a pig, or a sheep) and harvesting and analyzing an optic nerve from the animal. In some cases, an optic nerve is harvested at 1, 2, 3, 4, 5, 6, 7, 14, and/or 21 days post-transplant. In some cases, static analysis of remyelination of harvested optic nerves is accomplished using histology, confocal microscopy, and/or cross-sectional electron microscopy.

Robust survival and myelination of chambered axons by induced pluripotent stem cell-derived oligodendrocyte precursor cells can require a compromise between the nutrient and support factor needs of the axons, the oligodendrocyte precursor cells, and the maturing oligodendrocytes. In some cases, media conditions necessary to maintain healthy axons and fluid dynamics involved in media replenishment and ongoing provision of growth factors can be optimized. In some cases, fluidic shear stress during the addition of factors or cells to microfluidic chambers can be minimized. In some cases, by exploiting the microfluidic separation of the cell body chamber from one or more axon chambers, different media formulations can be introduced into the separate chambers and oligodendrocyte precursor cells survival and differentiation are increased. In some cases, timing of OPC introduction into the axon chambers is optimized and standardized. In some cases, frequency of media replenishment and the chemical components of the axon chamber media formulation can be optimized and standardized. In some cases, chamber media for OPC differentiation can include: DMEM/F12 w/o HEPES or phenol red; 1.25X B-27 Supplement, serum free; 0.25X N1 Medium Supplement; 0.5X N-2 Supplement; 30 ng/mL T3; 27.5 μM 2-mercaptoethanol; 2.5 ng/mL NT3; 50 ng/mL biotin; 0.5 μM dibutyryl cAMP; 2.5 ng/mL PDGF-AA; 2.5 ng/mL IGF-1; and 5 ng/mL BDNF. In some cases, methods and materials provided herein can be used to test factors, e.g., mitogens, signaling molecules, and oligo-inductive factors, for their effect on OPC differentiation. Examples of such factors include, without limitation, those listed in Table 1.

TABLE 1
Experimental factors for testing in microfluidic chambers

| Factor | Function |
|---|---|
| Noggin | Promotes oligodendrocyte differentiation and maturation |
| Netrin-1 | Promotes oligodendrocyte process extension & branching |
| Neuregulin | Enhances myelination of axons, OLs switch to NMDA receptor-dependent |
| | myelination |
| CNTF | Enhances oligodendrocyte survival |
| IL-11 | Potentiates oligodendrocyte survival, maturation, and myelin formation |
| EGF | Increases OPC generation, migration from the subventricular zone, and |
| | proliferation |
| FGF-2 | O4-positive OPC generation from CD44-positive cells, proliferation |
| Erythropoietin | Increases maturation, survival, and myelin production |
| Hydrocortisone | Enhances oligodendrocyte survival |
| Ascorbic Acid | Induces myelination |
| N-acetyl-L- | Enhances oligodendrocyte survival |
| cysteine | |
| Semaphorin 3F | Increases OPC recruitment and survival |
| Vitamin D | Enhances OPC differentiation via retinoid X receptor γ (RXR-γ) |
| Wnt, Axin-2 | Promotes β-catenin degradation, allowing differentiation of OPCs |
| CDK5 | Promotes OPC maturation and myelination |
| TGFβ | Enhance viability, promotes proliferation & maturation |
| Activin B | Enhances viability and maturation |
| TIMP-1 | Promotes differentiation and myelination |
| PDGF/T3 balance | Promotes differentiation |

In some cases, tested and optimized culture conditions can be assessed for efficacy as compared to mouse OPCs derived by shake-off from mixed glia cultures (i.e. non-iPSC-derived). In some cases, myelination is assessed by: 1) confocal and super-resolution microscopy and quantification of 3D reconstructed GFP-positive membrane structures, 2) transmission EM and analysis of myelin wrapping morphology, including g ratio, 3) RTPCR analysis of myelin gene expression levels in the axon chamber, and/or 4) Western blot analysis of myelin protein expression levels in the axon chamber. In some cases, optimized and standardized media conditions can increase the remyelination potential quotient of a population of induced pluripotent stem cell-derived oligodendrocyte precursor cells.

In some cases, quantitative robustness of the remyelination potential quotient of a population of induced pluripotent stem cell-derived oligodendrocyte precursor cells can be assessed by altering the density of iPSC-derived OPCs added to the axon chamber in order to systematically change the ratio of OPCs to axons (ROTA). In some cases, the remyelination potential quotient of a population of induced pluripotent stem cell-derived oligodendrocyte precursor cells can be determined by measuring the RPQ when the ROTA is sufficient to maximize, but not exceed, the axonal space available for myelination.

In some cases, maturation or myelination efficiency of an induced pluripotent stem cell-derived oligodendrocyte precursor cell cultured in the presence of a factor or factors can be increased compared to maturation or myelination efficiency of an induced pluripotent stem cell-derived oligodendrocyte precursor cell cultured in the absence of a factor or factors.

The invention will be further described in the following examples, which do not limit the scope of the invention described in the claims.

### EXAMPLES

### Example 1—Experimental Procedures

### Preparation of Microfluidic Chambers

MFCs were prepared as described elsewhere (Sauer et al., Neurobiol. Dis., 59:194-205 (2013)). Briefly, silicone elastomer (Sylgard 184) base and curing agent (mixed 10:1) were poured over etched fused silica molds. MFCs were placed in a vacuum chamber and incubated at 37° C. before being cut out from the molds and sterilized for use. In a sterile hood, acid washed and sterile coverslips (22×22 mm) were placed in 6-well tissue culture plates and coated with 0.5 mg/mL poly-ornithine overnight at 37° C. Prior to plating cells, the printed surface of the sterilized MFCs was adhered to glass cover slips to achieve a leak-proof chamber.

### Primary Cortical Neurons Grown in MFCs

Primary murine cortical neuron cultures were prepared as described elsewhere (Sauer et al., Neurobiol. Dis., 59:194-205 (2013)). Briefly, cells were obtained from embryonic day 15 B6 mouse cortices and plated at 1.5×10⁵ cells per microfluidic chamber with half media changes on alternate days. After verification of axonal growth through the microgrooves (day in vitro (DIV) 4-5)), aliquots of OPCs to be tested were plated into the middle chamber of the MFC and co-cultured for up to two weeks with differentiation factors.

### Immunocytochemistry

Following fixation in the MFC with 4% paraformaldehyde, cells were stained to determine the extent of myelination. Cells were incubated with blocking buffer (5% serum of secondary-antibody host species, 1% BSA, 0.1% Triton-X in DPBS containing Ca2+ and Mg2+) for 60 minutes at room temperature followed by overnight incubation with primary antibodies at 4° C. (PLP: Millipore, MAB388, 1:200; MOG: Millipore, MAB5680 1:100; MBP: Millipore, MAB386, 1:150; APC/CC1: Abcam, ab15270, 1:250; NF: Sternberger, SMI-312R, 1:750). After extensive washing, cells were stained with appropriate secondary antibodies, counterstained with DAPI, and mounted onto glass slides.

### Mouse Model of Demyelination

Cuprizone is a copper chelator, which causes apoptosis of mature oligodendroglia, followed by microglial recruitment, and phagocytosis of myelin. Demyelination was induced by feeding mice a diet containing 0.3% cuprizone for 9 weeks. When fed on this diet, mice exhibit demyelination in a well-characterized series of events. Peak demyelination occurred at 6-7 weeks with spontaneous remyelination occurring 2-4 weeks after transition to regular diet. Efficacy of OPC transplants upon reintroduction of regular diet was evaluated. Assessment of remyelination following transplant was performed as described elsewhere (Deb et al., PLoS One, 5:e12478 (2010)). Briefly, semi-thin sections of optic nerves were stained with para-phenylenediamine, imaged at 100× magnification following a predetermined sampling scheme, and analyzed using automated ImageJ macros to measure myelinated fibers.

### Transplant Injections

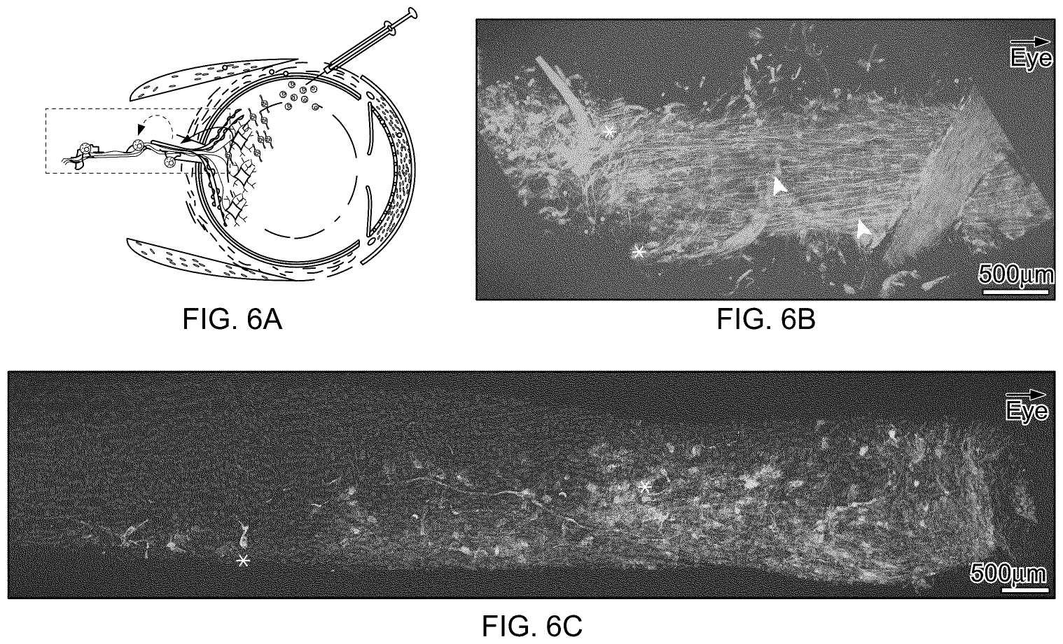

Maintaining a sterile field, 1.0 μL of media containing >10⁴ OPCs were injected into the vitreous humor of anesthetized mice, using a custom 34-gauge needle mounted on a 5 μL Hamilton syringe. Despite the small dimensions of the murine eye and the high needle gauge, OPCs were consistently and reproducibly delivered into the vitreous cavity with this method, and transferred cells were imaged using fluorescent fundus imaging of the mouse eye (evidence from >30 mice; data not included) (FIG. 6A).

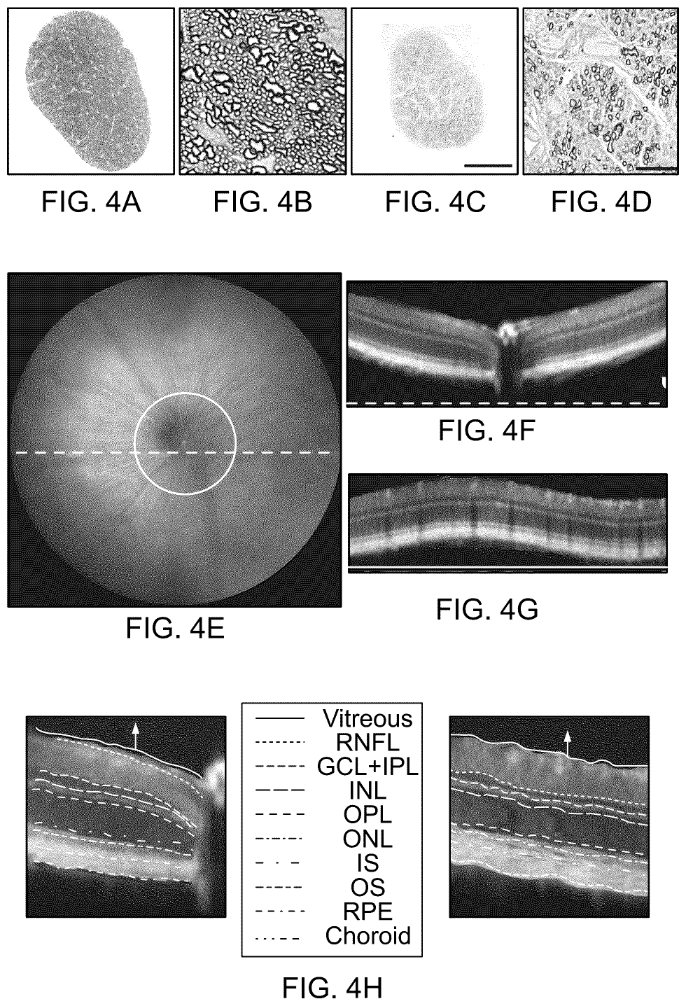

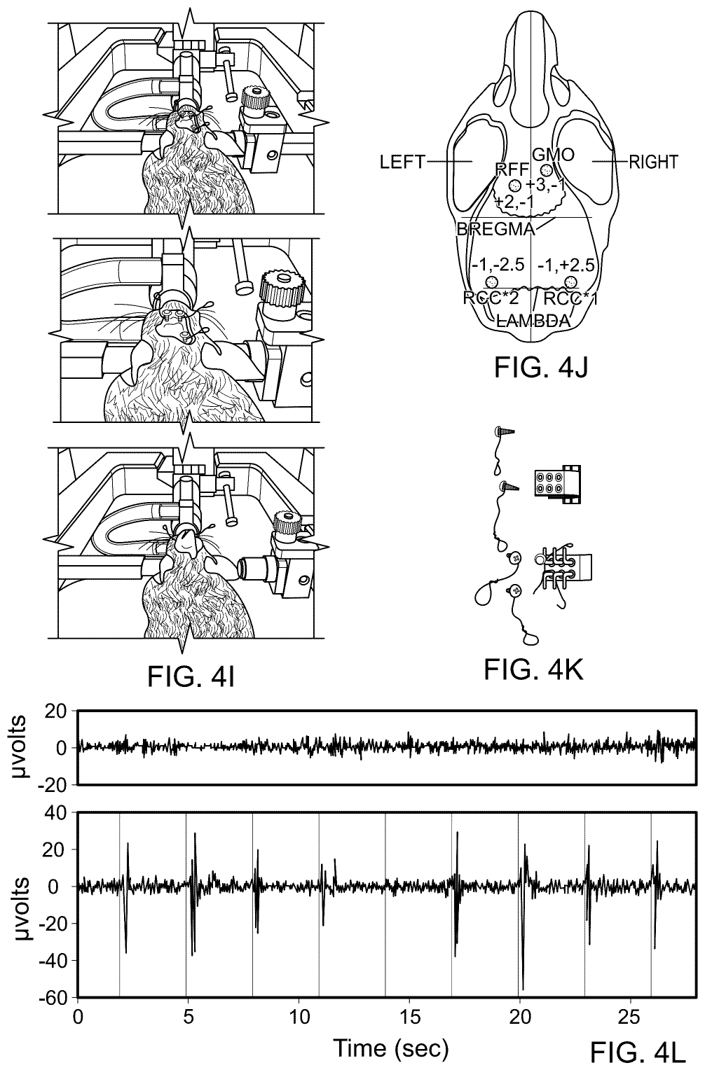

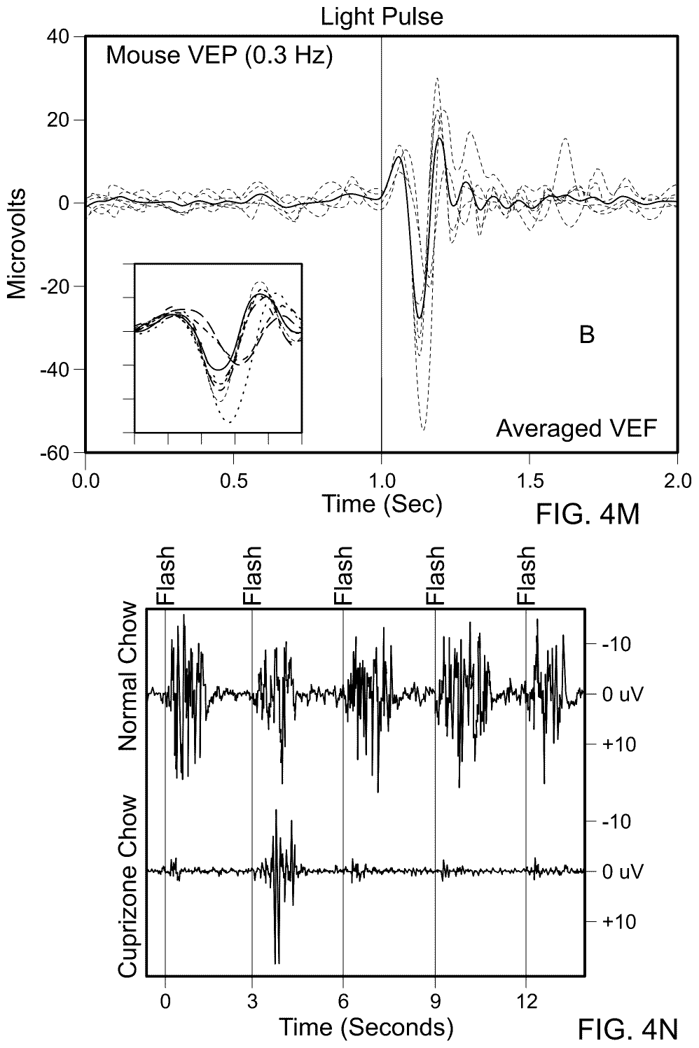

Image-guided optical coherence tomography (OCT): OCT was an imaging technique based on light interferometry which can be used to capture images from biomedical tissue at micrometer resolution. An image guided OCT system (Micron III, Phoenix Labs, USA) with a resolution of 4 μm, similar to human OCT scanners available today, was used. The system had the capability to capture bright-field as well as 3 channel fluorescent retinal images (FIG. 4E, 6B, 6C).

### Visual Evoked Potentials (VEPs)

To measure the functional outcomes of OPC transplantation, VEPs were recorded. Intracranial supradural screw electrodes (plastics1.com 8L0X3905201F) were implanted over the visual cortex as described elsewhere (Deb et al., (2010) PLoS One 5:e12478) (FIG. 4I-4K). After dark adaptation, mice were placed in a recording chamber connected to a recording system (8200-K1-SE3, Pinnacle Technology) using an implanted EEG headmount (8235-SM, Pinnacle Tech) tethered to a preamplifier (8202-SE3, Pinnacle Tech) and an analog-to-digital converter. Mice were visually stimulated by an on-axis flashing LED. VEPs were recorded with varying stimulus blocks from 0.1 Hz to 1000 Hz (controlled via a programmable Arduino Diecimila). Recording parameters measured latency to N1 (the first negative peak following stimulation), amplitude range between Nₘₐₓ-Pₘₐₓ (first positive peak after stimulation), number of stimuli not followed by a response, duration of visual evoked response, pulse width of Pₘₐₓ. Any delay in conduction velocity through the optic nerve increased time to N1, whereas axonal degeneration led to decrease in amplitude range, and an increase in response duration as well as increased pulse width due to non-synchronous firing of neurons, providing a very sensitive measure of optic nerve function.

### Tissue Collection

For collection of optic nerves and brain tissue, mice were perfused with 4% paraformaldehyde or Trump's fixative, and CNS tissues were post-fixed in the respective solution for 24 hours.

### Immunohistochemistry

Following adequate fixation, whole optic nerves and retinas were transferred to a 48 well dish and blocked for 2 hours in PBS containing 5% serum of secondary-antibody host species, 1% BSA, and 0.1% Triton-X. For tissues intended for clearing and whole tissue imaging, the amount of Triton-X was raised to 1%. After blocking, optic nerves were incubated overnight at 4° C. with primary antibodies (MOG: Millipore, MAB5680 1:100; MBP: Millipore, MAB386, 1:150; APC: Abcam, ab15270, 1:250). Tissues were washed extensively, stained for 2 hours with appropriate secondary antibodies, counterstained with DAPI, and mounted onto glass slides for microscopy or moved to clearing reagents.

### Clearing

Optic nerves were cleared using a modification of a ScaleSQ protocol as described elsewhere (Hama H et al., (2015) Nat Neurosci 18:1518-1529). Briefly, tissues were placed in dextro-sorbitol (25% w/v), urea (9.5 M), and Triton-X 100 (3% w/v) in distilled water at 37° C. overnight and subsequently in dextro-sorbitol (40% w/v), urea (4M), glycerol (15% w/v), and DMSO (20% v/v) in distilled water for 5 hours. After visual verification of tissue clarity, optic nerves were mounted on glass cover slides for image acquisition.

### Microscopy

Fluorescence and bright field images of cultured cells and slide-mounted whole retina and optic nerve tissues were imaged with a LSM 780 inverted confocal microscope, an upright two photon microscope (Olympus FV1000MPE), or an inverted Axio Observer Z1 microscope equipped with Apotome.

### Example 2—Microfluidic Chamber Design

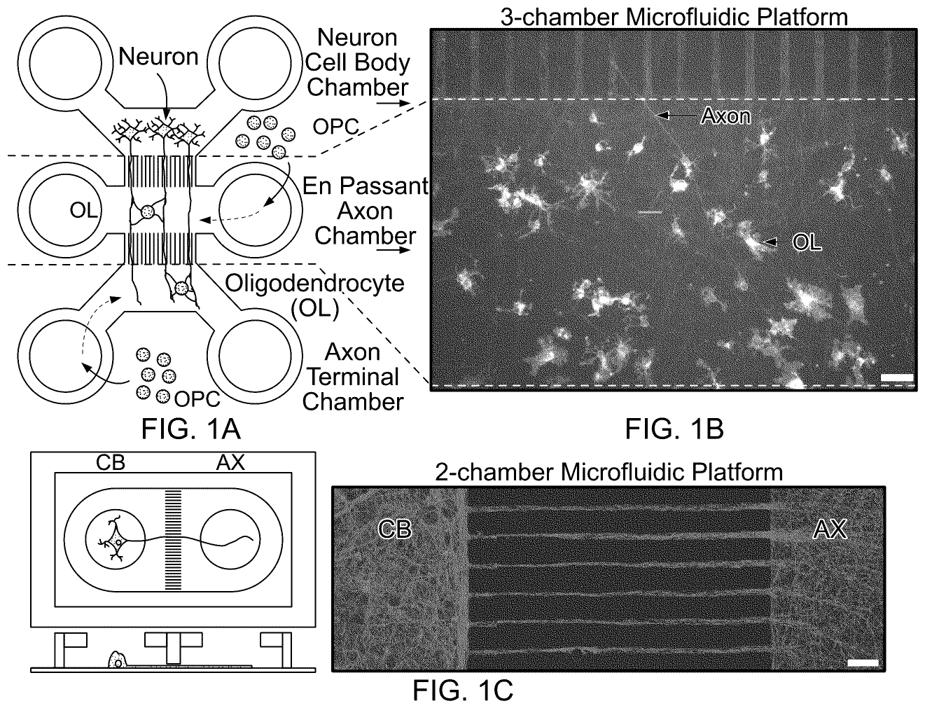

Murine cortical neurons were successful cultured in microfluidic chambers in order to isolate the neuronal cell bodies from their axons. Microgrooves within a microfluidic device prevented cell bodies from entering the axonal chamber, thereby allowing easier visualization and manipulation of axons without the interference of neuronal cell bodies and other cells types (such as oligodendrocytes and astrocytes) that are present at plating (FIG. 1C). A three-chamber microfluidic device was designed that not only allowed the separation of neuronal cell bodies and axons but permitted the addition of OPCs to a middle chamber that provided en passant access to the axons while minimizing fluidic shear stress (FIGS. 1A and 1B), thus allowing for easier quantification of in vitro myelination. The design shown in FIGS. 1A and 1B also permits the use of different combinations of growth and differentiation factors in order to optimize survival and maturation of OPCs and oligodendrocytes.

### Example 3—Directed Differentiation Protocol for Generating OPCs from Induced Pluripotent Stem Cells

Cell populations enriched for OPCs were created from murine lines as described elsewhere (Terzic et al., Cell Transplantation, 25(2):411-424 (2016)).

### Example 4—In Vitro Evidence of Oligodendrocyte Precursor Cell Maturation

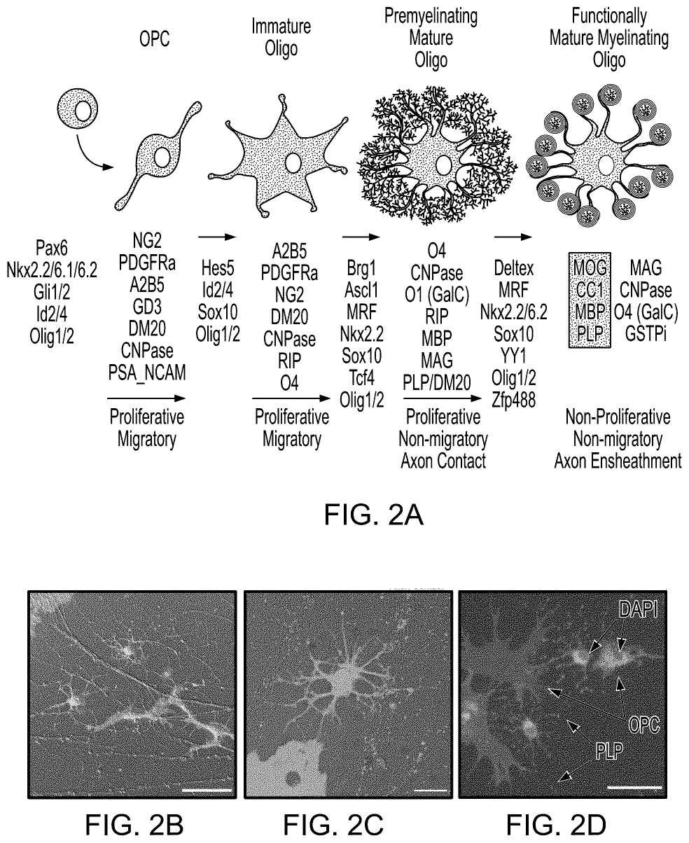

Utilizing a three-chamber MFC system, mouse iPSC-derived OPCs were successfully co-cultured with primary cortical neuron axons. The OPCs became GFP+ oligodendrocytes and a subset made extensive branching contacts with neuronal axons (FIGS. 2B and 2C), a morphological phenotype indicative of mature myelinating oligodendrocytes. Immunocytochemistry for proteolipid protein (PLP) (FIG. 2D) confirmed the ability of these oligodendrocytes to generate mature myelin.

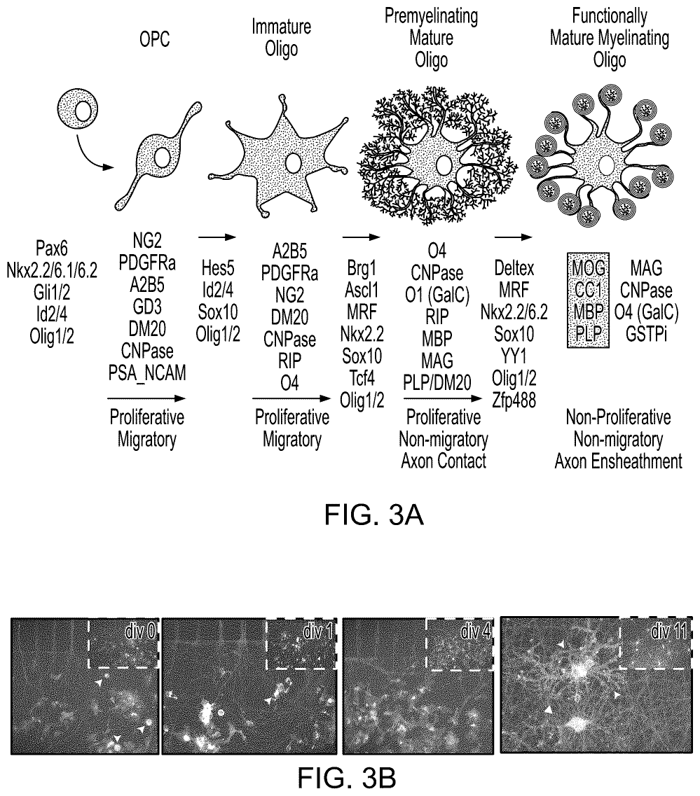

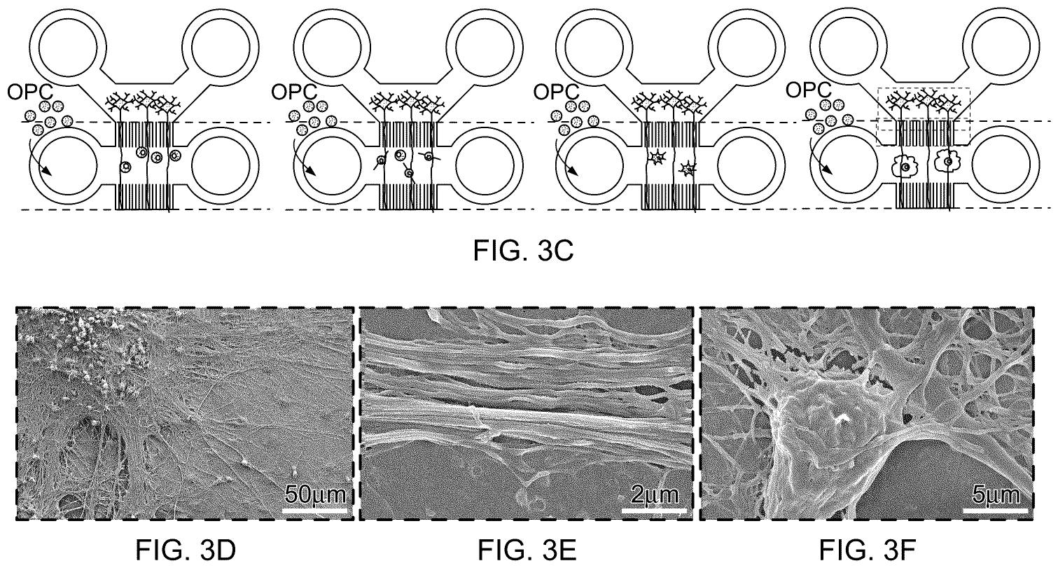

In a separate experiment utilizing a three-chamber MFC system, OPCs were co-cultured with neuronal axons for up to 14 days, and serial longitudinal imaging of OPCs was performed. By 11 days in culture, OPCs exhibited the phenotype of a mature oligodendrocyte, making multiple contacts with multiple axons (FIG. 3B). Transmission electron micrographs were taken of neuronal cell bodies extending axons in the proximal chamber of the microfluidic device (FIG. 3D), axon bundles passing through microgrooves connecting the proximal and middle chambers (FIG. 3E), and an oligodendrocyte extending processes making contact with multiple neuronal axons (FIG. 3F).

These results demonstrated successfully co-culturing iPSC-derived OPCs in a MFC three-chamber platform, and also demonstrated the ability to quantify the myelinating capacity of OPCs prior to transplantation.

### Example 5—Cuprizone-Induced Demyelination Model and Live Animal Assessments

Cuprizone is a copper chelator that causes apoptosis of mature oligodendroglia, followed by microglial recruitment and phagocytosis of myelin. Demyelination was induced by feeding mice a diet containing 0.3% cuprizone for 9 weeks. When fed on this diet, mice exhibit demyelination in a well-characterized series of events. Peak demyelination occurs at 6-7 weeks with spontaneous remyelination occurring 2-4 weeks after transition to regular diet. PPD staining of thin sections from araldite-embedded optic nerve revealed the pattern of normal myelination in the optic nerve of animals fed control chow (FIGS. 4A-B) and the profound demyelination observed after 6 weeks of cuprizone treatment (FIG. 4C-D). Optical coherence tomography (OCT) provided quantitative assessment of neuroretinal integrity. The retinal layers measured along the green line in FIG. 4E in a healthy mouse (FIG. 4F). Note the clear retinal nerve fiber layer (RNFL), as outlined in FIG. 4H. The retinal layers measured along the yellow line in FIG. 4E in a healthy mouse (FIG. 4G). Note the thick ganglion cell layer (GCL), as outlined in FIG. 4H. Head mount surgery for placement of VEP electrodes and recording locations (FIG. 4I-K). Visual evoked potentials (VEPs) measured in the visual cortex induced by a 0.3 Hz light pulse (FIG. 4L). The top trace represents unstimulated potentials, the bottom trace shows the potentials evoked by repetitive stimulation. VEP averaging is used to establish measurements of evoked potential amplitude, latency, and pulse width (FIG. 4M). VEP recording from visual cortex shows increased latency to N1 at 0.3 Hz stimulation frequency and increased failure to transmit impulses in the cuprizone-fed animal (bottom trace) compared to animals on regular diet (top trace) (FIG. 4N). Scale bar in FIG. 4C=100 μm and refers to FIG. 4A; scale bar in FIG. 3D=10 μm and refers to FIG. 4B.

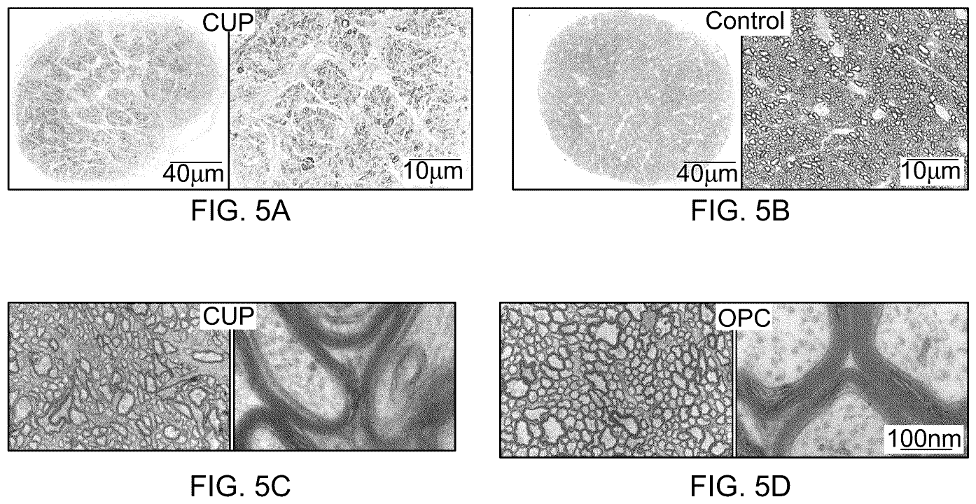

Semi-thin sections from araldite-embedded optic nerves were stained with PPD (p-phenylenediamine). The staining revealed the pattern of normal myelination in the optic nerve of animals fed control chow (FIG. 5B) and the profound demyelination observed after 9 weeks of cuprizone treatment (FIG. 5A). Electron microscopy of thin optic nerve cross sections was performed, and revealed thinning, and disorganization of myelin layers in cuprizone treated animals (FIG. 5C). Compacted myelin was restored two weeks after OPC transplant injection (FIG. 5D).

### Example 6—Intravitreal Transplantation of Induced Pluripotent Stem Cell-Derived Oligodendrocyte Precursor Cells

To further validate the in vitro findings above, GFP+ OPCs were transplanted into the vitreous cavity of mice (FIG. 6). In hosts with demyelinated optic nerves, transplanted cells produced long GFP+ fibers suggestive of myelin sheaths.

### Example 7—Functional Assessment of Remyelination In Vivo

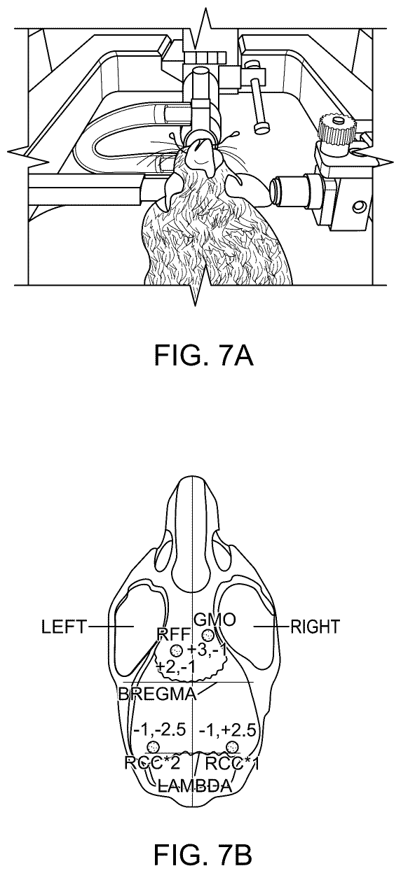

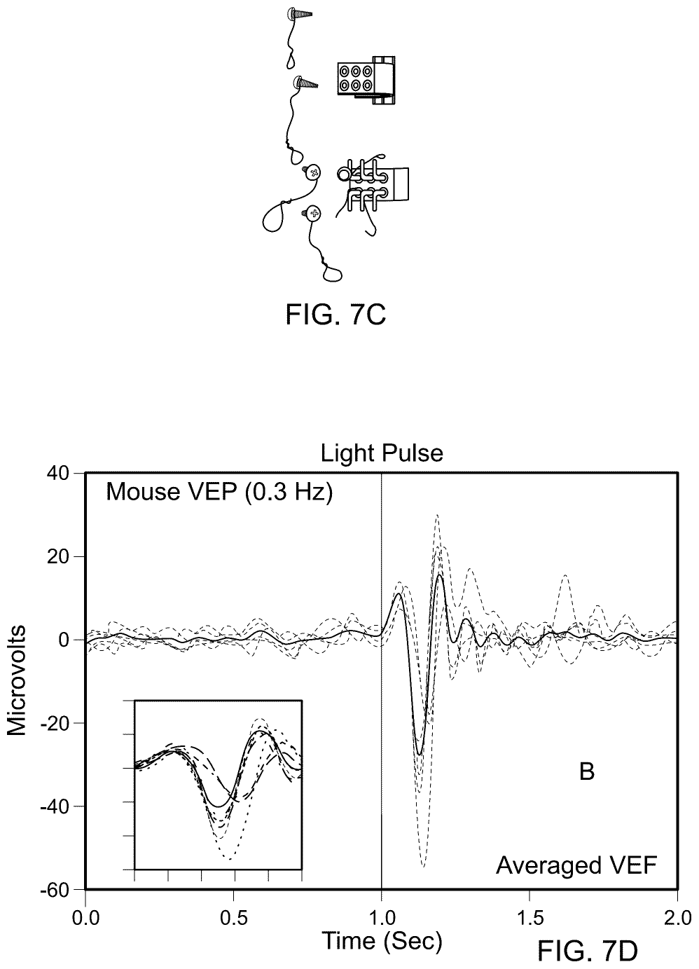

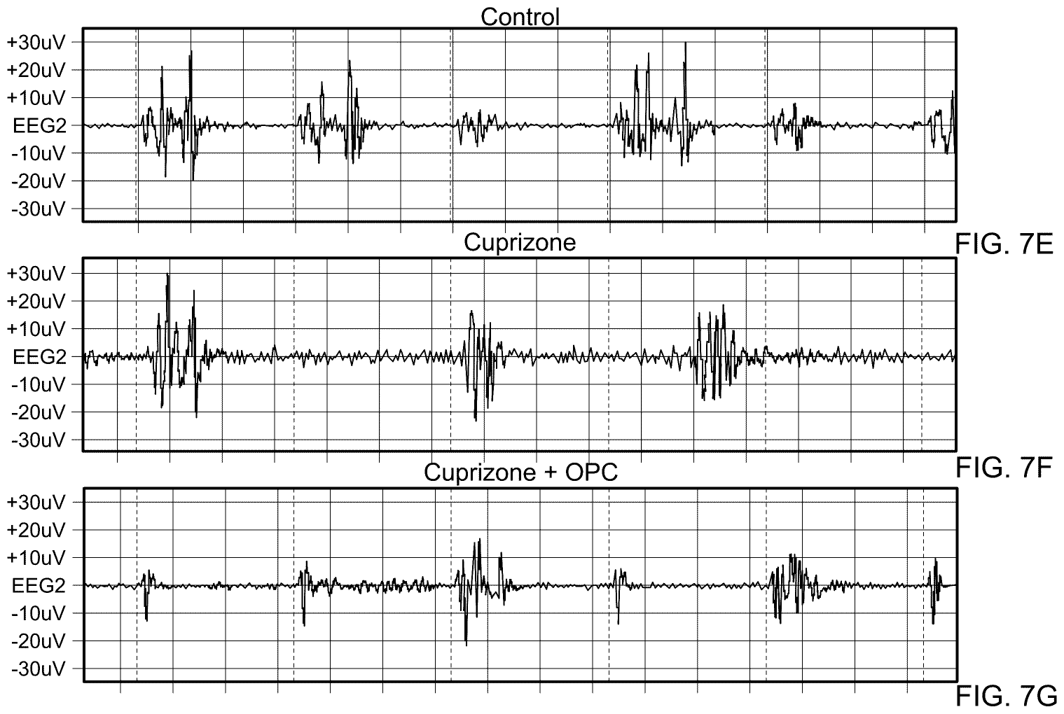

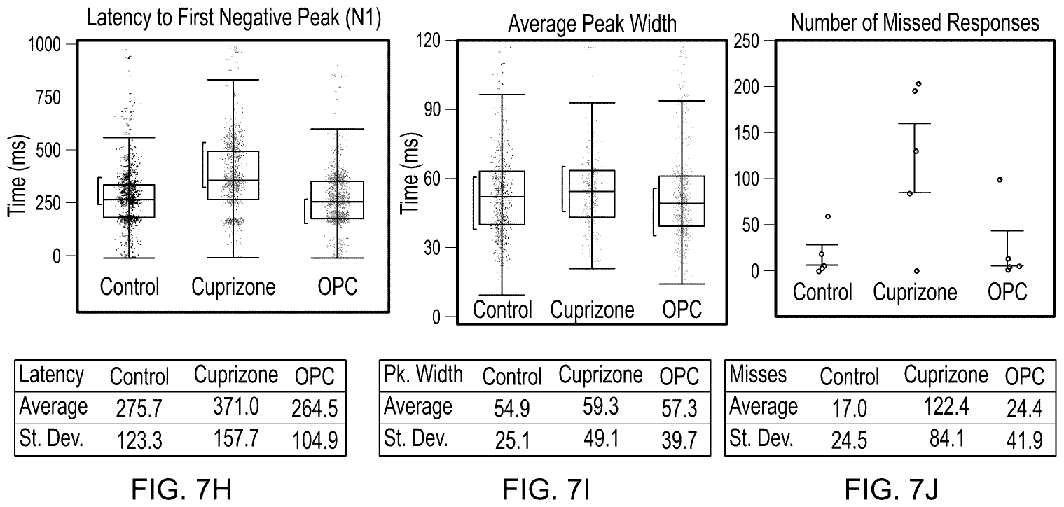

Mice were subjected to electrode implantation surgery and either received OPC transplant or served as controls (FIGS. 7A-C), and visual evoked potential (VEP) averaging was used to establish measurements of evoked potential amplitude, latency, and peak pulse-width (FIG. 7D). Representative waveforms of VEP recordings in a healthy control animal (FIG. 7E), cuprizone-fed animal (FIG. 7F), and cuprizone-fed animal transplanted with OPCs (FIG. 7G). Latency was measured as the time from the visual stimulus to the first negative peak in the VEP impulse (FIG. 7H). An increased latency indicated decreased conduction velocity as a result of loss of myelin and Nodes of Ranvier. The average latency increased from 276 ms to 371 ms after cuprizone treatment for nine weeks, and normalized to 265 ms after OPC transplant treatment. Peak width was measured as the duration of the first positive impulse at half-maximum amplitude. An increase is peak width denoted unsynchronized impulses travelling to the visual cortex at different speeds along axons with differing loss of myelin and conduction velocities. The shortest half for peak width values increased after demyelination with cuprizone, and returned towards baseline after OPC transplant (FIG. 7I). The number of missed responses represents the number of times a fixed stimulus did not evoke a response. The number of missed responses was greatly increased in cuprizone fed animals, and re-normalized after OPC transplant (FIG. 7J). All analysis was completed using MATLAB (MathWorks, MA, USA). n=5 for all groups.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a damaged optic nerve in a mammal, comprising:
a) identifying said mammal as having a condition of the optic nerve comprising optic nerve demyelination,
b) identifying a population of induced pluripotent stem cell-derived oligodendrocyte precursor cells as having a remyelination potential quotient greater than about 25 percent, and
c) administering said population of induced pluripotent stem cell-derived oligodendrocyte precursor cells to said mammal.

2. The method of claim 1, wherein said mammal is a human.

3. The method of claim 1, wherein said population is identified as having a remyelination potential quotient greater than about 25 percent by culturing a first portion of said population of induced pluripotent stem cell-derived oligodendrocyte precursor cells in a microfluidic device comprising first and second microfluidic chambers,
wherein said first microfluidic chamber comprises a neuron cell body of a cortical neuron,
wherein said second microfluidic chamber comprises an axon of said cortical neuron, and
wherein said first portion of said population of induced pluripotent stem cell-derived oligodendrocyte precursor cells is co-cultured with said axon in said second microfluidic chamber.

4. The method of claim 3, wherein said population is identified as having a remyelination potential quotient greater than about 25 percent by determining the number of cells of said first portion of said population of induced pluripotent stem cell-derived oligodendrocyte precursor cells having a characteristic of a mature, myelinating oligodendrocyte and dividing said number of cells of said first portion by the number of induced pluripotent stem cell-derived oligodendrocyte precursor cells introduced into said second microfluidic chamber.

5. The method of claim 4, wherein said characteristic of a mature, myelinating oligodendrocyte is selected from the group consisting of: a morphological characteristic, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, expression of a GST-pi mRNA, and combinations thereof.

6. The method of claim 4, wherein said remyelination potential quotient is determined to be sufficient for administration of said population of induced pluripotent stem cell-derived oligodendrocyte precursor cells to said mammal if said remyelination potential quotient is about 30 percent or higher.

7. The method of claim 3, wherein said microfluidic device further comprises a third microfluidic chamber,
wherein said second microfluidic chamber comprises a segment of said axon,
wherein said third microfluidic chamber comprises a distal end of said axon, and
wherein a second portion of said population of induced pluripotent stem cell-derived oligodendrocyte precursor cells is co-cultured with said distal end of said axon in said third microfluidic chamber.

8. The method of claim 7, wherein said population of induced pluripotent stem cell-derived oligodendrocyte precursor cells is identified as having a remyelination potential quotient greater than about 25 percent by determining the number of cells of said first portion of said population of induced pluripotent stem cell-derived oligodendrocyte precursor cells having a characteristic of a mature, myelinating oligodendrocyte and dividing said number of cells of said first portion by the number of induced pluripotent stem cell-derived oligodendrocyte precursor cells introduced into said second microfluidic chamber.

9. The method of claim 8, wherein said characteristic of a mature, myelinating oligodendrocyte is selected from the group consisting of: a morphological characteristic, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, expression of a GST-pi mRNA, and combinations thereof.

10. The method of claim 8, wherein said remyelination potential quotient is determined to be sufficient for administration of said population of induced pluripotent stem cell-derived oligodendrocyte precursor cells to said mammal if said remyelination potential quotient is about 30 percent or higher.

11. The method of claim 7, wherein said population of induced pluripotent stem cell-derived oligodendrocyte precursor cells is determined to have a remyelination potential quotient greater than about 25 percent by determining the number of cells of said second portion of said population of induced pluripotent stem cell-derived oligodendrocyte precursor cells having a characteristic of a mature, myelinating oligodendrocyte and dividing said number of cells of said second portion by the number of induced pluripotent stem cell-derived oligodendrocyte precursor cells introduced into said third microfluidic chamber.

12. The method of claim 11, wherein said characteristic of a mature, myelinating oligodendrocyte is selected from the group consisting of: a morphological characteristic, expression of a MOG polypeptide, expression of a CC1 polypeptide, expression of a MBP polypeptide, expression of a PLP polypeptide, expression of a MAG polypeptide, expression of a GST-pi polypeptide, expression of a MOG mRNA, expression of a CC1 mRNA, expression of a MBP mRNA, expression of a PLP mRNA, expression of a MAG mRNA, expression of a GST-pi mRNA, and combinations thereof.

13. The method of claim 11, wherein said remyelination potential quotient is determined to be sufficient for administration of said population of induced pluripotent stem cell-derived oligodendrocyte precursor cells to said mammal if said remyelination potential quotient is about 30 percent or above.

14. The method of claim 1, wherein said administering comprises intravitreal injection of said population of induced pluripotent stem cell-derived oligodendrocyte precursor cells.

15. The method of claim 14, wherein said mammal has a condition comprising multiple sclerosis, demyelinating optic neuritis, or both.

16. The method of claim 15, wherein said administering drives remyelination of the optic nerve, restores axonal conduction, or both.
